(19) United States
(12) Patent Application Publication
(10) Pub. No.: US 2010/0082038 A1
(43) Pub. Date: Apr. 1, 2010

(54) APPARATUS AND METHOD FOR PERFORMING AN INTRASTROMAL ABDOMINAL TRACHELECTOMY

(76) Inventor: DARYOOSH SAMIMI, Fountain Valley, CA (US)

Correspondence Address: JAFARI LAW GROUP, P.C., 801 N. PARKCENTER DRIVE, SUITE 220, SANTA ANA, CA, 92705, US

(21) Appl. No.: 12/239,222

(22) Filed: Sep. 26, 2008

(51) Int. Cl. A61B 17/42

(52) U.S. Cl. 606/119

(57) ABSTRACT

A method for performing an intrastromal abdominal trachelectomy, which is comprised of the steps of inserting a cutting guide into a female cervix, anchoring said cutting guide within said cervix, and making a plurality of incisions within said cervix using said cutting guide. In one embodiment, an apparatus for performing an intrastromal abdominal trachelectomy, which is comprised of a cutting guide for use in making an incision in a female cervix, an elongated member adapted to be inserted into said cervix, and a stabilizer that is adapted to be anchored onto a tissue of said cervix.

### TECHNICAL FIELD OF THE INVENTION

The present invention relates in general to an apparatus and method for performing an intrastromal abdominal trachelectomy as a bloodless nerve-sparing procedure without disrupting the pelvic support system. More specifically, the invention relates to a device and method for performing an intrastromal abdominal trachelectomy that generally reduces the risk of cervical cancer, reduces hospital stay, and reduces post-operation complications, such as post-operation infections, ureter injuries, vaginal vault prolapse and post-trachelectomy fistula.

### BACKGROUND OF THE INVENTION

Cervical cancer is said to be the second most common cancer found worldwide among women, the third most common cause of cancer-related deaths, and the most common cause of mortality from a gynecologic malignancy. Some of the causes of cervical cancer can be attributed to other types of medical surgical procedures performed within the female reproduction anatomy.

For instance, one of the relatively common procedures performed today is a supracervical hysterectomy. A supracervical hysterectomy is one alternative of a hysterectomy that may be performed in order to treat medical conditions such as benign fibroid tumors of the uterus, ovarian cysts, endometriosis, abnormal uterine bleeding and chronic pelvic pain without affecting urinary continence or sexual function. As a result, in a supracervical hysterectomy, the uterus is removed but leaves the cervix in place.

The resulting disadvantage of a supracervical hysterectomy is that often a treating physician will find that a patient who had this procedure done in the past has now developed a cervical disease in the cervical stump. Some of the problems that arise from the residual cervical stump are pain, bleeding, necrosis, recurrence of fibroids, and the development of cancer. In fact, the supracervical hysterectomy procedure has been criticized in medical literature due to the potentially fatal cancer that can develop in the cervical stump.

Another disadvantage associated with the supracervical hysterectomy is that it is expensive due to the cancer preventative measures that are taken into account. For example, there are varying preventive measures a doctor may recommend or prescribe to a patient who has undergone a supracervical hysterectomy that has left a cervical stump.

When a doctor determines that the removal of the patient's cervix is the best option, the patient will have to undergo a trachelectomy in order to remove the cervix. One method of performing a trachelectomy is an abdominal trachelectomy. This is considered within the medical community as a conventional method of performing a trachelectomy.

In the conventional abdominal trachelectomy, a surgeon may remove the cervix by cutting the uterosacral ligaments, the cardinal ligament of Mackenrodt, and the uterine vessel, all before entering the vaginal fornix. Once the ligaments are cut, the cervix is then severed from the vagina in a circular manner at the cervico-vaginal junction. Typically, in order to access the cervico-vaginal junction, the bladder is pushed downwards, or is even dissected free of its attachments.

As a result of this conventional trachelectomy, significant damage occurs to the Franken Hauser's nerve plexus, the vesical plexus, and other downstream nerves. Also a problematic result is that fibrous condensation in the endopelvic fascia is severed and is no longer able to support the vaginal vault.

Furthermore, in the conventional trachelectomy, substantial blood loss may occur as well as other post-operative complications, such as infections, ureter injuries, vaginal vault prolapses and post-trachelectomy fistula.

There is a need in the art for a device and procedure to perform a trachelectomy without severing the surrounding nerves and without disturbing the pelvic support system. Specifically, there is a need for a device and method that provides a way to make a precise incision in the cervical area in order to remove the cervix without severing or adversely affecting any of the surrounding nerve structures, reproductive organs, or other surrounding anatomical systems. There is also a need for an apparatus and method to perform a trachelectomy that does not result in post-operation infections, ureter injuries, vaginal vault prolapses and post-trachelectomy fistula. There is also a need for an apparatus and method for performing a trachelectomy that does not result in significant blood loss or the necessity of a transfusion. There is also a need for an apparatus and method for performing a trachelectomy that does not result in a prolonged hospital stay. There is also a need for an apparatus and method for performing a trachelectomy that can prevent or reduce the likelihood of the occurrence of cervical cancer.

### SUMMARY OF THE INVENTION

To minimize the limitations in the prior art, and to minimize other limitations that will be apparent upon reading and understanding the present specification, the present invention describes an apparatus and method for performing an intrastromal abdominal trachelectomy as a nerve sparing procedure that reduces harm or effects on the pelvic support system.

An apparatus for performing an intrastromal abdominal trachelectomy, in accordance with the present invention, comprises of a cutting guide for use in making an incision in a female cervix, an elongated member adapted to be inserted into said cervix, and a stabilizer that is adapted to be anchored onto a tissue.

A method for performing an intrastromal abdominal trachelectomy, in accordance with the present invention, that comprises the steps of inserting a cutting guide into a female cervix, anchoring said cutting guide within said cervix, and making a plurality of incisions within said cervix using said cutting guide.

It is an objective of the present invention to facilitate and perform an intrastromal abdominal trachelectomy with the claimed device as a bloodless nerve-sparing method without disturbing the pelvic support system.

It is another objective of the present invention to perform an intrastromal abdominal trachelectomy with the claimed device as an alternative procedure to prevent blood loss and the need for transfusions.

It is another objective of the present invention to perform an intrastromal abdominal trachelectomy with the claimed device to enable a shorter hospital stay.

It is another objective of the present invention to perform an intrastromal abdominal trachelectomy with the claimed device in order to prevent post-operative conditions, such as post-operation infections, ureter injuries, vaginal vault prolapses and post-trachelectomy fistula.

It is another objective of the present invention to perform an intrastromal abdominal trachelectomy with the claimed device in order to prevent cervical cancer.

It is another objective of the present invention to perform an intrastromal abdominal trachelectomy with the claimed device that maintains the cardinal and uterosacral ligaments, nerve fibers, and pericervical ring as un-severed and intact.

Finally, it is yet another objective of the present invention to provide a way to perform an intrastromal abdominal trachelectomy with a device, as claimed, that helps to identify the anatomy during the surgical procedure.

These and other objectives, advantages and features of the present invention are described herein with specificity so as to make the present invention understandable to one of ordinary skill in the art.

### BRIEF DESCRIPTION OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

FIG. 1 is a side perspective view of the claimed apparatus that is used for performing an intrastromal abdominal trachelectomy in an exemplary embodiment of the invention. In this view, the side of the cutting guide connected to the elongated member is herein depicted. On the opposite side of the cutting guide is attached a deployment handle for use in insertion of the apparatus during the trachelectomy. At the end of the elongated member is positioned a stabilizer for use in anchoring the entire apparatus within the cervix in order to maintain its position during the trachelectomy.

FIG. 2 illustrates a cross-sectional view of a pericervical ring of a cervix near a cervico-uterine junction where the claimed device is inserted vaginally into the cervical canal. The cutting guide is positioned proximate the center of the pericervical ring to act as guide when making an incision within the cervical stroma for purposes of ultimately removing the cervix.

FIG. 3 illustrates a front view of the female reproductive anatomy showing where the claimed device is inserted as used in the intrastromal abdominal trachelectomy. In order to prevent against severing or otherwise adversely affecting the surrounding nerves or supporting ligaments, the claimed device, when used during the intrastromal abdominal trachelectomy, is able to assist in identifying the anatomy.

### DETAILED DESCRIPTION OF THE DRAWINGS

In the following discussion that addresses a number of embodiments and applications of the present invention, reference is made to the accompanying drawings that form a part hereof, where depictions are made, by way of illustration, of specific embodiments in which the invention may be practiced. It is to be understood that other embodiments may be utilized and changes may be made without departing from the scope of the present invention.

Before beginning a discussion of the diagrams included herein, it is asserted that the claimed device was tested in a group of twenty patients, all of whom had an intrastromal abdominal trachelectomy performed. Each of the twenty patients was operated on by the same doctor at the same institution. The length of the intrastromal abdominal trachelectomy was measured from the point at which the claimed device was applied to a patient. For each of the patients, the following parameters were evaluated: (1) pre-operative and post-operative hemoglobin levels, (2) number of days for hospital stay, (3) debility morbidity, (4) wound healing, and (5) readmission to the hospital due to ureter injury, vaginal prolapse, or post-trachelectomy fistula.

In general, there were differences in the average blood loss and average hospital stay. Overall, the intrastromal abdominal trachelectomy proved to show reduced blood loss as well as shorter hospital stays. In addition, no post-operation infections, ureter injuries, vaginal vault prolapses, or post-trachelectomy fistula were seen to have resulted in any of the patients operated upon. Due to the reduced blood loss in the patients, no transfusions were needed for any of the patients. Based on this group of patients, the use of the claimed device in the intrastromal abdominal trachelectomy appears to be an effective alternative for the traditional trachelectomy that is currently performed in patients.

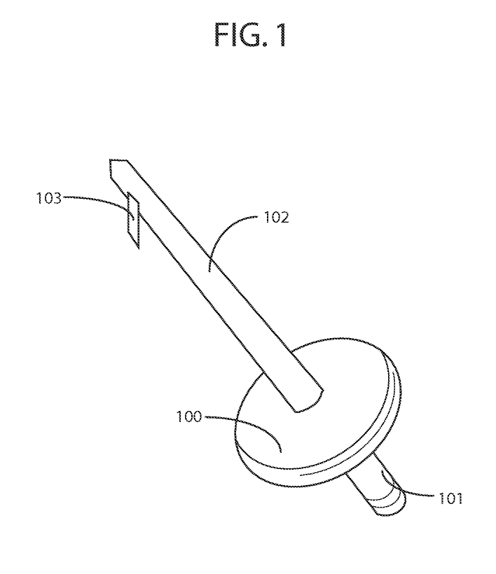

Now turning to the figures, FIG. 1 illustrates a side perspective view of the claimed device that is used for performing an intrastromal abdominal trachelectomy. One side of a disc-like shaped cutting guide 100 is shown facing upwards. Cutting guide 100 is coupled to a deployment handle 101 on one of its side (not seen in this figure) and an elongated member 102 on its other side (depicted herein). The elongated member is coupled to a stabilizer 103 near its end opposite of where the cutting guide 100 is positioned.

The deployment handle 101 can be used for inserting the cutting guide 100, elongated member 102, and stabilizer 103 into a cervix (not depicted) during an intrastromal abdominal trachelectomy. The deployment handle 101 assists in being able to properly guide the device into the cervix. The deployment handle 101 may be square or rectangular in shape with a series of parallel-positioned square-shaped grooves engraved therein. The square-shaped grooves may be engraved within the deployment handle 101 for purposes of attaining a better grip whether used manually or in conjunction with a medical instrument or machine, such as a clamp.

The deployment handle 101 may vary in size, and may have the following measurements of an approximate length of 1.2 centimeters (cm) and an approximate width of 0.6 cm. While instrumental in placement of the device, the deployment handle 101 is not an essential feature of the device. In fact, other comparable ways of guiding and/or handling the device into the cervix may be used and incorporated into the device without limiting the scope of the invention.

The cutting guide 100 is disc-shaped, having a substantially circular shape, and is coupled with said deployment handle 101. The exact measurements of the cutting guide 100 may vary in size; however, the cutting guide 100 may be approximately 0.3 cm in thickness and approximately 2 cm in diameter. Other shapes, such as an elliptical shape, may be employed for the cutting guide 100.

The measurements specified herein are not intended to limit the scope of the invention. It may be necessary to vary the size of the cutting guide as well as other of the device's components as appropriate. This is because a female's reproductive anatomy may differ slightly in size or shape. Typically, a cervix, or the mouth of the uterus, may range in size from 2.5 cm to 3 cm. However, a woman might have an unusually small or large cervix. For a reproductive-aged woman, her cervical canal will typically have a certain range of measurements. In addition, in a parous woman, the cervix is usually bulkier and the cervix's external os may appear wider. Thus, it may be necessary from a medical standpoint to adjust the claimed device's shape and dimension as needed for certain patients.

As explained above, the elongated member 102 is coupled to the opposite side of the cutting guide 100 that is coupled to the deployment handle 101. The length of the elongated member 102 may be approximately 4.2 cm. Thus, the entire length of the device from the end of the elongated member 102 to the end of the deployment handle may be approximately 5.7 cm. Overall, the elongated member 102 has a generally cylindrical shape. The end of the elongated member 102 coupled to the cutting guide 100 has a substantially cylindrical shape with an approximate inner diameter of 0.6 cm. As one follows the length of the elongated member 102 towards its other end, it becomes tapered in shape, forming a tip at its most distal end. At this distal end, the elongated member 102 may have an inner diameter of approximately 0.3 cm. The tip-shape at the distal end of the elongated member 102 is also a spherical radius-like shape. All parts of the claimed device, with the exception of the stabilizer 103, may be made of plastic, in accordance with an exemplary embodiment of the invention.

Although it is not an absolute requirement of the claimed device, acetal plastic is a suitable material for making the claimed device due its properties, which are usually characterized by a good fatigue life, low moisture sensitivity, high resistance to solvents and chemicals, and good electric properties. It should be noted that other comparable materials may be used to make the claimed device without deviating from the scope of the claimed invention.

The stabilizer 103 serves the purpose of providing an anchoring or a locking function for the claimed device once it is inserted into the cervix in order to maintain its position during the trachelectomy. In an exemplary embodiment, the stabilizer 103 is positioned at the distal end of the elongated member 102 at approximately two-thirds the length of the elongated member 102 from the cutting guide 100. The stabilizer 103 may be located approximately 1 cm from the spherical radius-shape end. The stabilizer 103 may be approximately 1 millimeter (mm) in diameter and approximately 1 cm in length. The stabilizer is positioned so that it is obliquely situated on the elongated member 102, which can be at an approximate 30 degree angle. The end of the stabilizer 103 that is slanted upwards in the 30 degree angle generally faces in the direction of the cutting guide 100.

The stabilizer 103 may be positioned at varying angles, such as 10 degrees or 15 degrees. However, if the stabilizer 103 is positioned at an angle of more than 30 degrees, there exists the potential for a laceration in the cervix tissue to occur when the claimed device is in use.

The stabilizer 103 is made of a slightly flexible steel material and is integrated into the claimed device when it is formed using any convention injection molding and curing technique. It is not required that the stabilizer 103 be coated with plastic since when used in a 30 degree angle, no damage occurs to the tissue of the cervical canal when the stabilizer is anchored therein.

The flexible steel material of the stabilizer 103 may serve as a radio-opaque marker for purposes of locating the stabilizer 103 should it become lost or detached from the claimed device. The elongated member 102 may also contain a plurality of stabilizers in accordance with any medical concerns or other factors the doctor or surgeon may take into account with respect to anchoring the device in the cervix.

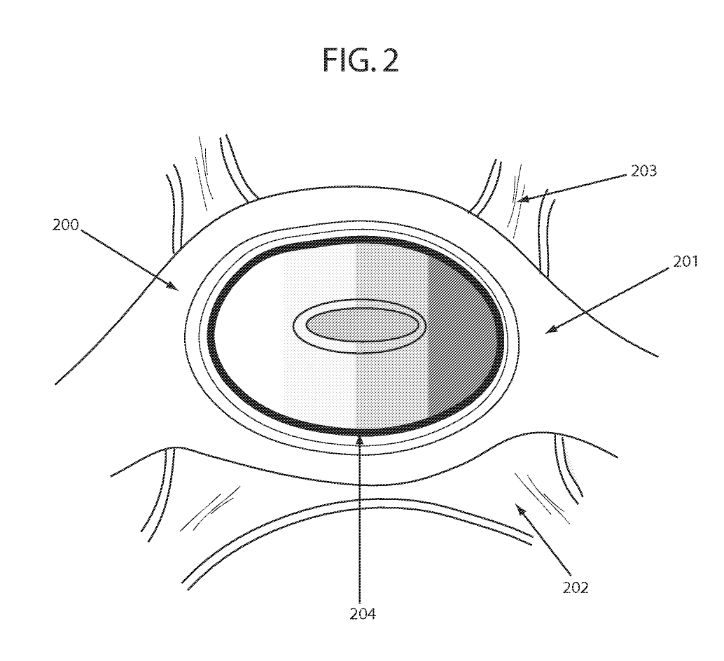

FIG. 2 illustrates in a cross-sectional view at the cervico-uterine junction where the main anatomical feature, the pericervical ring 200 of the cervix, is located. It is at the pericervical ring 200 where the claimed device is used to begin the actual removal of the cervix. Even though the pericervical ring 200 is an anatomical location involved in the use of the claimed device, it is left intact and is not incised or otherwise separated or detached from the cervix or its surrounding support structures.

In fact, the claimed device assists in protecting the pericervical ring 200 during the intrastromal abdominal trachelectomy so that the surrounding nerve structures and pelvic support system are not significantly disturbed. As previously discussed, the cardinal ligament 201 and the uterosacral ligament 202 are the main support structures of the cervix. Also depicted in FIG. 2 is the vesico-cervical ligament 203, which is also attached to the peripheral portion of the cervix. When a pericervical incision 204 is made within the pericervical stroma (not shown), the surrounding ligaments 201, 202 and 203 remain substantially intact and undisturbed through the trachelectomy. The peripheral portion of the cervix should be protected since it is fused with this ligamentous support system and other nerves. Use of the claimed device protects the integrity of these ligaments in order to avoid the adverse results like those seen in the traditional abdominal trachelectomy.

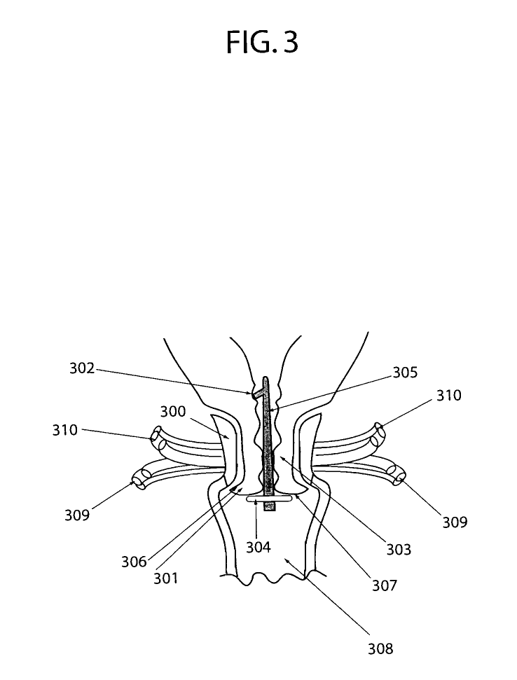

FIG. 3 illustrates a front view of the female reproductive anatomy showing where the claimed device is inserted as used in the intrastromal abdominal trachelectomy. In order to prevent against severing or otherwise adversely affecting the surrounding nerves or supporting ligaments, the claimed device, when used during the intrastromal abdominal trachelectomy, is able to assist in identifying the anatomy.

Before commencing use of the claimed device, an adequate amount of vasoconstrictive solution is injected into the peripheral cervix 300 until ischemia is attained. Then the claimed device is inserted vaginally through the cervical canal 301 beginning with the portion of the claimed device containing the stabilizer 302 toward the internal os of the cervix 303 until the cutting guide 304 can no longer be pushed inwards.

In this diagram, only one stabilizer 302 is depicted. However, as previously discussed in FIG. 1, a plurality of stabilizers may be integrated into the elongated member 305. The plurality of stabilizers may be placed in various positions along the length of the elongated member 305.

The cutting guide 304 acts as guide when making an incision in the cervical canal 301 and cervical stroma 306 for purposes of ultimately removing the cervix 303. Once the cutting guide 304 is in place, a thermoelectric or laser knife is used to begin making the surgical incisions from the abdominal side or above. The dissection into the cervix 303 should be performed in a substantially circular manner. Once one whole circular incision is made around the upper side of the cervical stroma 306, the claimed device is adjusted, in order to continue making a series of continuous circular incisions. Each incision in the series of circular incisions is made in a gradual manner and in intervals of approximately 0.5 cm at a time. By gradually performing the circular incisions in small intervals, bleeding is reduced.

The incisions should not be made in the fascia of the anterior, posterior or lateral sides of the cervix 303, rather the incisions should be made within the cervical stroma 306. The gradual incisions made within the cervical stroma 306 allow for the cervix 303 to ultimately be removed from within the pericervical ring 307. As mentioned, the claimed device acts as an identifier of the anatomy throughout the process of making each incision in the cervix 303. Incisions are made until the vagina 308 is reached. Once all of the necessary incisions are made, the entire cervix 303 is removed from within the pericervical ring 307 without disturbing the surrounding support ligaments or nerves.

In FIG. 3, one can see the surrounding ligaments, specifically the cardinal ligament 309 and the uterosacral ligament 310, which are attached to the peripheral portion of the cervix 303. When the cervix 303 is removed with the claimed device, the cardinal ligament 309 and the uterosacral ligament 310 still remain substantially intact and undisturbed so as not to result in some of the adverse effects that can occur in the traditional trachelectomy.

Once the cervix 303 is removed, the surgeon is to begin making the necessary surgical closures. In order to close the pericervical ring 307, a continuous interlocking method with delay absorbable sutures are to be made starting from the vaginal margin. It should be noted that the closure of the vaginal fornix can be accomplished by using the same method of making such sutures. The remaining closures should be continued from the vaginal side toward cephalic by use of a spiral or tornado technique.

Once the upper margin of the pericervical ring 301 is closed, the surgical end of the round ligaments are to be anchored to the middle portion of the pericervical ring. By anchoring the surgical end of the round ligament, this provides for additional pelvic support. Any remaining raw surfaces should be carefully peritonealized during closure of the abdominal area.

An apparatus and method for performing an intrastromal abdominal trachelectomy has been described. The foregoing description of the various exemplary embodiments of the invention has been presented for the purposes of illustration and disclosure. It is not intended to be exhaustive or to limit the invention to the precise form disclosed. Many modifications and variations are possible in light of the above teaching. It is intended that the scope of the invention not be limited by this detailed description, but by the claims and the equivalents to the claims.

What is claimed is:

1. An apparatus for performing an intrastromal abdominal trachelectomy comprising:
a cutting guide for use in making an incision in a female cervix;
an elongated member adapted to be inserted into said cervix; and
a stabilizer adapted to be anchored onto a tissue.

2. The apparatus of claim 1, wherein said cutting guide has two opposite sides.

3. The apparatus of claim 2, wherein said cutting guide is integrally coupled on its first side with a deployment handle.

4. The apparatus of claim 3, wherein said cutting guide is integrally coupled on its second side with said elongated member.

5. The apparatus of claim 4, wherein said elongated member is integrally coupled with said stabilizer.

6. The apparatus of claim 5, wherein said stabilizer is positioned laterally on a distal end of said elongated member that is opposite said cutting guide.

7. The apparatus of claim 6, wherein said elongated member and stabilizer are guided into said cervix by use of said deployment handle toward an internal os region of said cervix.

8. The apparatus of claim 7, wherein said tissue is a middle portion of a cervical canal of said cervix.

9. The apparatus of claim 8, wherein said stabilizer is adapted to be attached onto said middle portion of said cervical canal so as to hold said cutting guide in place.

10. The apparatus of claim 9, wherein said cutting guide is adapted to be situated on a center area of a pericervical ring of said cervix.

11. The apparatus of claim 10, wherein said cutting guide defines a substantially circular shape for incision within said cervical canal without severing said pericervical ring.

12. The apparatus of claim 11:
wherein said cutting guide has a width ranging from 0.2 cm to 0.3 cm and a diameter ranging from 1.7 cm to 2.3 cm;
wherein said elongated member is substantially cylindrical in shape and has a length ranging from 4.0 cm to 4.4 cm and an inner diameter ranging from longitudinally from 0.3 cm to 0.6 cm; and
wherein said deployment handle is substantially cylindrical in shape and has a length ranging from 1.0 cm to 1.5 cm and a diameter ranging from 0.4 cm to 0.8 cm.

13. The apparatus of claim 12, wherein said stabilizer:
has a length ranging from 0.8 cm to 1.2 cm;
is positioned between 0.8 cm to 1.2 cm below the distal end of said elongated member that is opposite said cutting guide; and
is positioned at a 30 degree angle that faces in the direction of said cutting guide.

14. The apparatus of claim 13, wherein said cutting guide, said elongated member, said deployment handle are comprised of acetal plastic; and wherein said stabilizer is made of a flexible steel material that is coated with acetal plastic.

15. A method for performing an intrastromal abdominal trachelectomy, comprising the steps of:
inserting a cutting guide into a female cervix;
anchoring said cutting guide within said cervix; and
making a plurality of incisions within said cervix using said cutting guide.

16. The method of claim 15, further comprising the step of steering said cutting guide into said cervix by use of a deployment handle that is integrally coupled on a first side of said cutting guide;
wherein said cutting guide is integrally coupled to an elongated member on its second side, and
wherein said elongated member is integrally coupled with a stabilizer positioned laterally on a distal end of said elongated member that is opposite said cutting guide.

17. The method of claim 16, further comprising the step of inserting said elongated member and stabilizer into a cervical canal of said cervix from a vaginal region below said cervix toward an internal os region of said cervix.

18. The method of claim 17, further comprising the step of anchoring said cutting guide to maintain its position on said pericervical ring by attaching said stabilizer on a middle portion of said cervical canal region of said cervix.

19. The method of claim 18, further comprising the steps of:
making a plurality of substantially circular incisions around said cutting guide within said cervical canal until said vaginal region is reached; and
reducing blood loss by separately performing each said substantially circular incision in intervals of 0.5 cm.

20. The method of claim 19, further comprising the step of removing said cervix after making said plurality of substantially circular incisions within said cervical canal without having severed any of the surrounding nerves or pelvic support system of said pericervical ring.
